(19) United States
(12) Patent Application Publication
(10) Pub. No.: US 2024/0081614 A1
(43) Pub. Date: Mar. 14, 2024

(54) ENDOSCOPIC EXAMINATION SUPPORT APPARATUS, ENDOSCOPIC EXAMINATION SUPPORT METHOD, AND RECORDING MEDIUM

(71) Applicant: NEC Corporation, Tokyo (JP)

(72) Inventors: Hiroyasu SAIGA, Tokyo (JP); Tatsu KIMURA, Tokyo (JP)

(73) Assignee: NEC Corporation, Tokyo (JP)

(21) Appl. No.: 18/517,105

(22) Filed: Nov. 22, 2023

Related U.S. Application Data
(63) Continuation of application No. 18/555,166, filed on Jan. 1, 0001.

(30) Foreign Application Priority Data
Aug. 1, 2022 (WO) PCT/JP2022/029450

(51) Int. Cl. A61B 1/00

(52) U.S. Cl. CPC A61B 1/000094; A61B 1/00006; A61B 1/00045; A61B 1/00055

(57) ABSTRACT

In the endoscopic examination support apparatus, the image acquisition means acquires captured images during removal of an endoscope. The posture change estimation means estimates a relative posture change of an endoscope camera from the captured images. The distance estimation means estimates a distance between a surface of colon and the endoscope camera from the captured images. The intestinal tract direction estimation means estimates an intestinal tract direction of the colon based on the posture change and the distance. The calculation means calculates a direction in which the endoscope camera should be directed, based on the intestinal tract direction and the relative posture of the endoscope camera. The output means outputs a display image including the direction in which the endoscope camera should be directed, to a display device. The display image can be used for support such as user's decision making.

### CROSS-REFERENCE TO RELATED APPLICATION

This application is a Continuation of U.S. patent application Ser. No. 18/555,166 filed on Oct. 12, 2023, which is a National Stage Entry of PCT/JP2023/028001 filed on Jul. 31, 2023, which claims priority from Japanese Patent Application PCT/JP2022/029450 filed on Aug. 1, 2022, the contents of all of which are incorporated herein by reference, in their entirety.

### TECHNICAL FIELD

The present disclosure relates to processing of images relating to an endoscopic examination.

### BACKGROUND ART

When a doctor operates the endoscope, the elasticity of the endoscope and the softness and complex shape of the colon may cause the endoscope camera to move to an unexpected position and approach the wall of the colon. This may result in areas that are not observable on the surface of the colon, leading to oversight of lesions. Patent Document 1 proposes to provide an insertion system that presents a recommended method of insertion operation when inserting the medical endoscope into the object of insertion.

### PRECEDING TECHNICAL REFERENCES

### Patent Document

Patent Document 1: International Publication WO2018/069992

### SUMMARY

### Problem to be Solved

However, Patent Document 1 is directed to a method of insertion operation of the endoscope, and it cannot present the direction of the endoscope camera so as to appropriately perform the observation of organs at the time of the removal of the endoscope.

One object of the present disclosure is to present the direction of an endoscope camera suitable for the observation in an endoscopic examination.

### Means for Solving the Problem

According to an example aspect of the present invention, there is provided an endoscopic examination support apparatus comprising:
an image acquisition means configured to acquire captured images during removal of an endoscope;
a posture change estimation means configured to estimate a relative posture change of an endoscope camera from the captured images;
a distance estimation means configured to estimate a distance between a surface of colon and the endoscope camera from the captured images;
an intestinal tract direction estimation means configured to estimate an intestinal tract direction of the colon based on the posture change and the distance;
a calculation means configured to calculate a direction in which the endoscope camera should be directed, based on the intestinal tract direction and the relative posture of the endoscope camera; and
an output means configured to output a display image including the direction in which the endoscope camera should be directed, to a display device.

According to another example aspect of the present invention, there is provided an endoscopic examination support method comprising:
acquiring captured images during removal of an endoscope;
estimating a relative posture change of an endoscope camera from the captured images;
estimating a distance between a surface of colon and the endoscope camera from the captured images;
estimating an intestinal tract direction of the colon based on the posture change and the distance;
calculating a direction in which the endoscope camera should be directed, based on the intestinal tract direction and the relative posture of the endoscope camera; and
outputting a display image including the direction in which the endoscope camera should be directed, to a display device.

According to still another example aspect of the present invention, there is provided a recording medium recording a program, the program causing a computer to execute processing of:
acquiring captured images during removal of an endoscope;
estimating a relative posture change of an endoscope camera from the captured images;
estimating a distance between a surface of colon and the endoscope camera from the captured images;
estimating an intestinal tract direction of the colon based on the posture change and the distance;
calculating a direction in which the endoscope camera should be directed, based on the intestinal tract direction and the relative posture of the endoscope camera; and
outputting a display image including the direction in which the endoscope camera should be directed, to a display device.

### Effect

According to the present disclosure, it is possible to present the direction of an endoscope camera suitable for observation in an endoscopic examination.

### BRIEF DESCRIPTION OF THE DRAWINGS

FIG. 1 is a block diagram showing a schematic configuration of an endoscopic examination system.

FIG. 2 is a block diagram showing a hardware configuration of an endoscopic examination support apparatus.

FIG. 3 is a block diagram showing a functional configuration of an endoscopic examination support apparatus.

FIG. 4 shows an example of a direction in which an endoscope camera should be directed.

FIG. 5 is a diagram showing a display example of a calculation result.

FIG. 6 is a diagram showing another display example of the calculation result.

FIG. 7 is a diagram showing still another display example of the calculation result.

FIG. 8 is a diagram showing still another display example of the calculation result.

FIG. 9 is a flowchart of direction calculation processing of the endoscope camera by the endoscopic examination support apparatus.

FIG. 10 is a block diagram showing a functional configuration of an endoscopic examination support apparatus of a second example embodiment.

FIG. 11 is a flowchart of processing by the endoscopic examination support apparatus of the second example embodiment.

### EXAMPLE EMBODIMENTS

Preferred example embodiments of the present invention will be described with reference to the accompanying drawings.

### First Example Embodiment

[System Configuration]

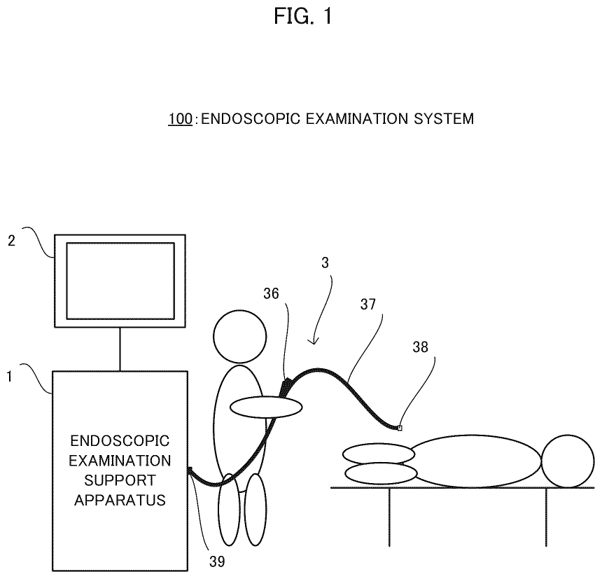

FIG. 1 shows a schematic configuration of an endoscopic examination system 100. The endoscopic examination system 100 estimates the direction of the intestinal tract and the direction of the endoscope camera during the endoscopic examination (including treatment). The endoscopic examination system 100 then presents a direction to direct the endoscope camera in the direction of the intestinal tract if the direction of the endoscope camera is not directed in the direction of the intestinal tract. A doctor can observe the entire intestinal tract by following the presentation of the endoscopic examination system 100 and directing the endoscope camera in the direction of the intestinal tract. Thus, it is possible to reduce the region that can not be observed.

As shown in FIG. 1, the endoscopic examination system 100 mainly includes an endoscopic examination support apparatus 1, a display device 2, and an endoscope 3 connected to the endoscopic examination support apparatus 1.

The endoscopic examination support apparatus 1 acquires a moving image (i.e., a video, hereinafter also referred to as an “endoscopic video Ic”) captured by the endoscope 3 during the endoscopic examination from the endoscope 3 and displays display data for the check by the examiner of the endoscopic examination on the display device 2. Specifically, the endoscopic examination support apparatus 1 acquires a moving image of the colon captured by the endoscope 3 as an endoscopic video Ic during the endoscopic examination. The endoscopic examination support apparatus 1 extracts frame images from the endoscopic video Ic, and estimates a distance between the surface of the colon and the endoscope camera (hereinafter also referred to as “depth”) and a relative posture change of the endoscope camera on the basis of the frame images. Then, the endoscopic examination support apparatus 1 performs three-dimensional restoration of the intestinal tract of the colon based on the depth and the relative posture change of the endoscope camera, and estimates the direction of the intestinal tract. The endoscopic examination support apparatus 1 estimates the direction in which the endoscope camera should be directed, based on the direction of the intestinal tract and the relative posture of the endoscope camera.

The display device 2 is a display or the like for performing a predetermined display on the basis of the display signal supplied from the endoscopic examination support apparatus 1.

The endoscope 3 mainly includes an operation unit 36 used by an examiner to input instructions such as air supply, water supply, angle adjustment, and an image-capturing instruction, a shaft 37 having flexibility and inserted into an organ of a subject to be examined, a tip portion 38 with a built-in endoscope camera such as an ultra-compact imaging element, and a connection unit 39 for connection with the endoscopic examination support apparatus 1.

[Hardware Configuration]

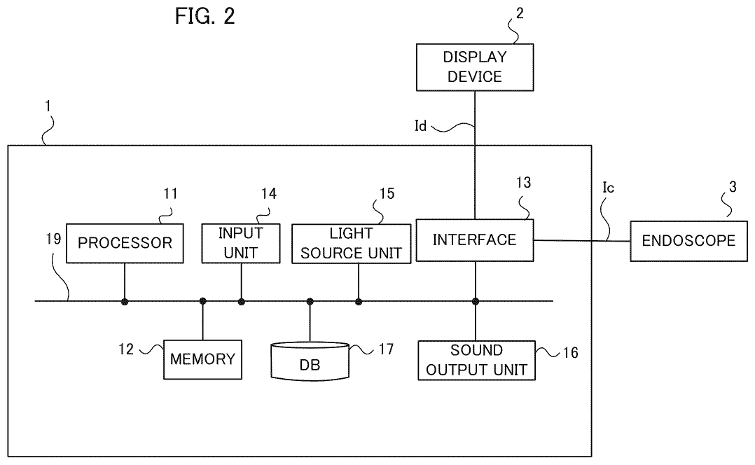

FIG. 2 shows a hardware configuration of the endoscopic examination support apparatus 1. The endoscopic examination support apparatus 1 mainly includes a processor 11, a memory 12, an interface 13, an input unit 14, a light source unit 15, a sound output unit 16, and a data base (hereinafter referred to as “DB”) 17. Each of these elements is connected via a data bus 19.

The processor 11 executes a predetermined processing by executing a program stored in the memory 12. The processor 11 is a processor such as a CPU (Central Processing Unit), a GPU (Graphics Processing Unit), and a TPU (Tensor Processing Unit). The processor 11 may be configured by a plurality of processors. The processor 11 is an example of a computer.

The memory 12 is configured by various volatile memories used as a working memory and non-volatile memories for storing information needed for processing the endoscopic examination support apparatus 1, such as a RAM (Random Access Memory) and a ROM (Read Only Memory). Incidentally, the memory 12 may include an external storage device such as a hard disk connected to or incorporated in the endoscopic examination support apparatus 1, and may include a storage medium such as a removable flash memory or a disk medium. The memory 12 stores a program for the endoscopic examination support apparatus 1 to execute each process in the present example embodiment.

Also, the memory 12 temporarily stores a series of endoscopic videos Ic captured by the endoscope 3 in the endoscopic examination, based on the control of the processor 11.

The interface 13 performs an interface operation between the endoscopic examination support apparatus 1 and the external devices. For example, the interface 13 supplies the display data Id generated by the processor 11 to the display device 2. Also, the interface 13 supplies the illumination light generated by the light source unit 15 to the endoscope 3. Also, the interface 13 supplies an electrical signal indicating the endoscopic video Ic supplied from the endoscope 3 to the processor 11. The interface 13 may be a communication interface such as a network adapter for wired or wireless communication with an external device, or may be a hardware interface compliant with a USB (Universal Serial Bus), SATA (Serial Advanced Technology Attachment), etc.

The input unit 14 generates an input signal based on the operation of the examiner. The input unit 14 is, for example, a button, a touch panel, a remote controller, a voice input device, or the like. The light source unit 15 generates light to be delivered to the tip portion 38 of the endoscope 3. The light source unit 15 may also incorporate a pump or the like for delivering water or air to be supplied to the endoscope 3. The sound output unit 16 outputs the sound based on the control of the processor 11.

The DB 17 stores the endoscopic images acquired by the previous endoscopic examination of the subject. The DB 17 may include an external storage device such as a hard disk connected to or incorporated in the endoscopic examination support apparatus 1, and may include a storage medium such as a removable flash memory. Instead of providing the DB 17 in the endoscopic examination system 100, the DB 17 may be provided in an external server or the like to acquire relevant information from the server through communication.

Incidentally, the endoscopic examination support apparatus 1 may be provided with a sensor, such as a magnetic sensor, which is capable of measuring the rotation and translation of the endoscope camera.

[Functional Configuration]

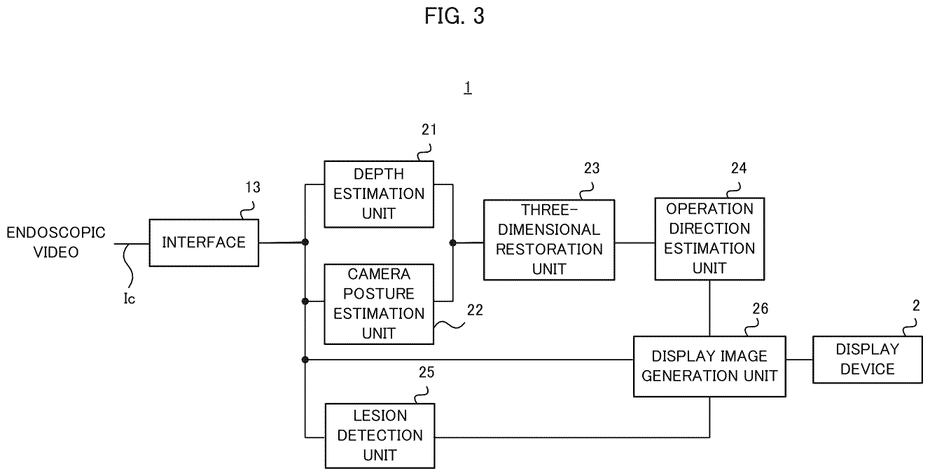

FIG. 3 is a block diagram showing a functional configuration of the endoscopic examination support apparatus 1. The endoscopic examination support apparatus 1 functionally includes the interface 13, a depth estimation unit 21, a camera posture estimation unit 22, a three-dimensional restoration unit 23, an operation direction estimation unit 24, a lesion detection unit 25, and a display image generation unit 26.

To the endoscopic examination support apparatus 1, the endoscopic video Ic is inputted from the endoscope 3. The endoscopic video Ic is inputted to the interface 13. The interface 13 extracts frame images (hereinafter, also referred to as “endoscopic images”) from the inputted endoscopic video Ic, and outputs the endoscopic images to the depth estimation unit 21, the camera posture estimation unit 22, and the lesion detection unit 25. Further, the interface 13 outputs the inputted endoscopic video Ic to the display image generation unit 26.

The endoscopic images are inputted from the interface 13 to the depth estimation unit 21. The depth estimation unit 21 estimates the depth from the inputted endoscopic images using an image recognition model prepared in advance or the like. Then, the depth estimation unit 21 outputs the estimated depth to the three-dimensional restoration unit 23.

To the camera posture estimation unit 22, the endoscopic images are inputted from the interface 13. The camera posture estimation unit 22 estimates the rotation and translation of the endoscope camera from the image-capturing point of a first endoscopic image to the image-capturing point of a second endoscopic image (i.e., the relative posture change of the endoscope camera, hereinafter simply referred to as “camera posture change”) using, for example, two successive endoscopic images in time. Then, the camera posture estimation unit 22 outputs the estimated camera posture change of the endoscope camera to the three-dimensional restoration unit 23. For example, the camera posture estimation unit 22 estimates the camera posture change from the inputted endoscopic images using an image recognition model or the like prepared in advance. It is noted that the camera posture estimation unit 22 may estimate the relative posture change of the endoscope camera using measurement data of a magnetic sensor.

Here, the image recognition models used by the depth estimation unit 21 and the camera posture estimation unit 22 are machine learning models trained, in advance, to estimate the depth and the camera posture change from the endoscopic images. These are also called “the depth estimation model” and “the camera posture estimation model”, respectively. The depth estimation model and the camera posture estimation model can be generated by so-called supervised learning.

For the training of the depth estimation model, teacher data in which the depth is given to the endoscopic image as a correct answer label is used, for example. The endoscopic images and depths used for the training are collected in advance from the endoscope camera and a ToF (Time of Flight) sensor provided in the endoscope. That is, the pairs of the RGB image taken by the endoscope camera and the depth are generated as the teacher data, and the teacher data are used for the training.

For the training of the camera posture estimation model, teacher data in which the posture change of the camera is given as a correct answer label to the endoscopic image is used, for example. In this case, the posture change of the camera can be acquired using a sensor that can detect rotation and translation, such as a magnetic sensor. That is, pairs of the RGB image taken by the endoscope camera and the posture change of the camera are generated as the teaching data, and the training is performed using the teaching data.

The teacher data used to train the depth estimation model and the camera posture estimation model may be created from a simulated image of the endoscope using CG (Computer Graphics). Thus, a large amount of teacher data can be created at high speed. The depth estimation model and the camera posture estimation model can be generated by the machine learning of the relationship between the endoscopic image and the depth/camera posture change.

The depth estimation model and the camera posture estimation model may be generated by self-supervised learning. For example, in self-supervised learning, motion parallax is utilized to create teacher data. Concretely, in self-supervised learning, a pair of the endoscopic image Iᵢ and the endoscopic image Iⱼ, a Depth CNN (Convolutional Neural Network) for estimating the depth from the endoscopic image Iᵢ, and a Pose CNN for estimating the relative posture from the endoscopic image Iᵢ and the endoscopic image iⱼ are prepared. Then, the endoscopic image Iⱼ (also called “endoscopic image I_{i→j}”) is reconstructed from the endoscopic image Iᵢ based on the depth and relative posture estimated by the Depth CNN and the Pose CNN. Then, the training of the model is performed using the difference between the reconstructed endoscopic image I_{i→j} and the actual endoscopic image Iⱼ as a loss.

The three-dimensional restoration unit 23 performs three-dimensional restoration processing of the intestinal tract based on the depth inputted from the depth estimation unit 21 and the relative posture change of the endoscope camera inputted from the camera posture estimation unit 22, and estimates the direction of the intestinal tract. Then, the three-dimensional restoration unit 23 outputs the three-dimensional model, the direction of the intestinal tract, the relative posture change of the endoscope camera, and the position of the endoscope camera to the operation direction estimation unit 24.

The operation direction estimation unit 24 receives the three-dimensional model, the direction of the intestinal tract, and the relative posture change of the endoscope camera from the three-dimensional restoration unit 23. Then, the operation direction estimation unit 24 calculates the direction in which the endoscope camera should be directed, based on the direction of the intestinal tract and the relative posture change of the endoscope camera. The operation direction estimation unit 24 outputs the three-dimensional model, the relative posture change of the endoscope camera, and the direction in which the endoscope camera should be directed, to the display image generation unit 26.

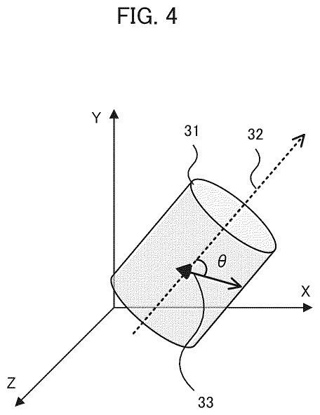

FIG. 4 shows an example of the direction in which the endoscope camera should be directed. In FIG. 4, a three-dimensional model 31 of the intestinal tract, an intestinal tract direction 32, and an endoscope camera direction 33 are shown on the XYZ coordinates. The three-dimensional model 31 is the model of the intestinal tract three-dimensionally restored by the three-dimensional restoration unit 23, and includes the detailed three-dimensional structure of the intestinal tract. However, in FIG. 4, for convenience of explanation, the three-dimensional model 31 is shown by approximating it to a cylindrical shape. The intestinal tract direction 32 is the longitudinal or axial direction of the intestinal tract, and is estimated based on the three-dimensional model 31 of the intestinal tract. The endoscope camera direction 33 is the direction of the lens of the endoscope camera, i.e., the shooting direction of the endoscopic camera.

In FIG. 4, the operation direction estimation unit 24 calculates the angle formed by the intestinal tract direction 32 and the endoscope camera direction 33, i.e., the deviation angle θ of the endoscope camera direction 33 with respect to the intestinal tract direction 32. When the deviation angle θ is equal to or larger than a predetermined threshold value, the operation direction estimation unit 24 determines that the endoscope camera is facing the intestine wall. When it is determined that the endoscope camera is facing the intestine wall, the operation direction estimation unit 24 calculates the direction in which the endoscope camera should be directed such that the direction of the endoscope camera coincides with the direction of the intestinal tract (i.e., such that the deviation angle θ becomes zero), and outputs the direction to the display image generation unit 26.

The endoscopic images are inputted from the interface 13 to the lesion detection unit 25. The lesion detection unit 25 detects the lesion candidate from the endoscopic images by using an image recognition model prepared in advance, and generates the lesion candidate image including the detected lesion candidate. The lesion detection unit 25 surrounds the lesion candidate on the lesion candidate image with an ellipse or the like, and outputs the lesion candidate image to the display image generation unit 26.

The display image generation unit 26 generates display data using the three-dimensional model, the relative posture change of the endoscope camera, the direction in which the endoscope camera should be directed, and the lesion candidate image, which are inputted from the operation direction estimation unit 24 and the lesion detection unit 25, and outputs the generated display data to the display device 2.

In the above-described configuration, the interface 13 is an example of an image acquisition means, the depth estimation unit 21 is an example of a distance estimation means, the camera posture estimation unit 22 is an example of a posture change estimation means, the three-dimensional restoration unit 23 is an example of an intestinal tract direction estimation means, the operation direction estimation unit 24 is an example of a calculation means, and the display image generation unit 26 is an example of an output means.

### Display Examples

Next, display examples by the display device 2 will be described.

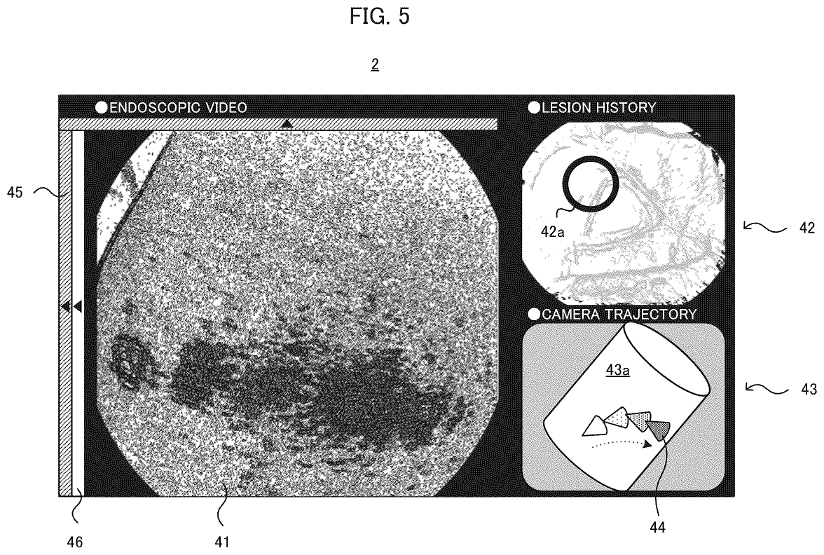

FIG. 5 is an example of a display by the display device 2. In this example, the display device 2 displays an endoscopic video 41, a lesion history 42, a camera trajectory 43, a camera mark 44, intestinal tract direction indicators 45, and a lesion direction indicator 46.

The endoscopic video 41 is the endoscopic video Ic during the examination and is updated as the endoscope camera moves. The lesion history 42 is an image indicating the detected lesion candidate in the endoscopic examination, and the lesion candidate image inputted from the lesion detection unit 25 is used. The lesion candidate area detected by the lesion detection unit 25 is shown by the circle 42a. Incidentally, when the lesion candidates are detected at multiple positions, an image of the most recent lesion candidate is displayed in the lesion history 42.

The camera trajectory 43 shows the trajectory of the endoscope camera within a predetermined time period. In FIG. 5, the three-dimensional intestinal tract model 43a is represented in a tubular shape, and the trajectory of the endoscope camera is indicated by superimposing and displaying the camera marks 44 indicating the orientation and position of the endoscope camera at predetermined times on the intestinal tract model 43a. The camera marks 44 schematically illustrate the orientation and position of the endoscope camera at different timings. In FIG. 5, the camera mark 44 is represented by a cone, and the bottom surface of the cone indicates the lens side of the endoscope camera. Also, the camera marks 44 are differently colored in time series, such that the darker color indicates the newer orientation and position of the endoscope camera. Incidentally, in FIG. 5, as indicated by the arrow, the camera direction of the endoscope camera is changing from the direction of the intestinal tract to the direction of the intestine wall.

The intestinal tract direction indicators 45 present the direction in which the endoscope camera should be directed, so as to direct the endoscope camera in the direction of the intestinal tract. The intestinal tract direction indicator 45 is displayed when the endoscope camera is facing the intestinal wall, specifically, when the aforementioned deviation angle θ is equal to or larger than the predetermined threshold value. In FIG. 5, the intestinal tract direction indicators 45 are displayed at the left and upper ends of the endoscopic video 41. Therefore, the doctor can know that the endoscope camera will be directed in the intestinal tract direction if the endoscope camera is directed to the left and upward direction. Incidentally, when the direction to which the endoscope camera should be directed is the right direction, the intestinal tract direction indicator 45 is displayed on the right end of the endoscopic video 41. When the direction to which the endoscope camera should be directed is the downward direction, the intestinal tract direction indicator 45 is displayed on the lower end of the endoscopic video 41. Thus, when the endoscope camera is facing the intestine wall, the intestinal tract direction indicator 45 is displayed at least one of the upper, lower, left and right ends of the endoscopic video 41, depending on the direction in which the endoscope camera should be directed.

On the other hand, the lesion direction indicator 46 presents the direction in which the endoscope camera should be directed, so as to direct the endoscope camera toward the lesion. The lesion direction indicator 46 is displayed when the lesion candidate is detected. In FIG. 5, the lesion direction indicator 46 is displayed at the left end of the endoscopic video 41. This allows the doctor to know that the endoscope camera will be directed toward the lesion candidate if the endoscope camera is directed to the left. Thus, when a lesion candidate is detected, the lesion direction indicator 46 is displayed at least one of the upper, lower, left and right ends of the endoscopic video 41, depending on the position of the lesion candidate.

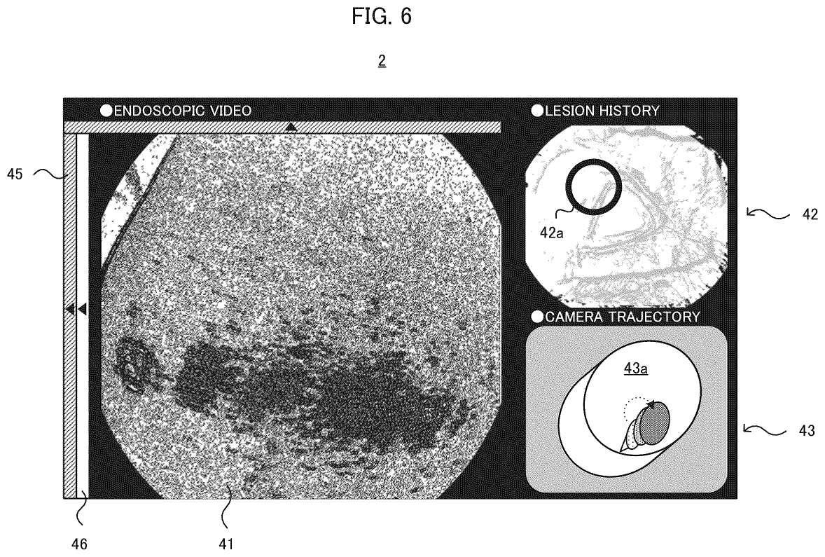

The display image generation unit 26 may generate the display data of the camera trajectory 43 so as to display the intestinal tract model 43a viewed in such a direction that the plurality of camera marks 44 overlap as little as possible. For example, in the example of FIG. 6, the camera trajectory 43 is displayed with the intestinal tract model 43a viewed in the direction of the intestinal tract. Therefore, the camera mark 44 overlaps with each other, and the doctor cannot properly grasp the trajectory of the endoscope camera. In this regard, the display image generation unit 26 determines, using principal component analysis or the like, the direction in which the dispersion of the camera direction indicated by the plurality of camera marks 44 is increased, and generates the display data for displaying the camera trajectory 43 in a state of viewing the intestinal tract model 43a in that direction. Thus, as shown in FIG. 5, the display device 2 can appropriately display the trajectory of the endoscope camera with the intestinal tract model 43a viewed in the direction in which the overlap of the camera mark 44 is small.

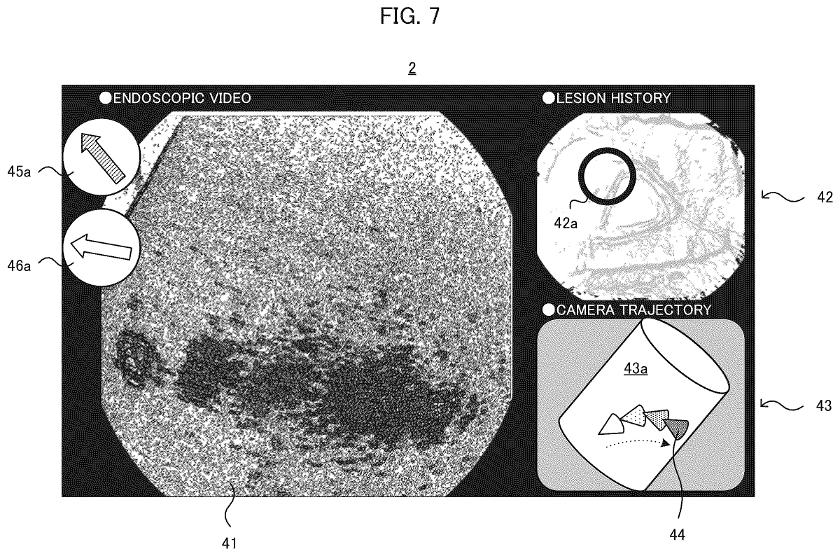

FIG. 7 shows another display example by the display device 2. This example is for the case in which the intestinal tract direction indicator and the lesion direction indicator are displayed by arrows. Specifically, in FIG. 7, the intestinal tract direction indicator 45a and the lesion direction indicator 46a are displayed on the endoscopic video 41. In FIG. 7, as compared with the case of arranging the indicators at the upper and lower ends and the left and right ends of the screen as shown in FIG. 5, a more detailed direction can be shown.

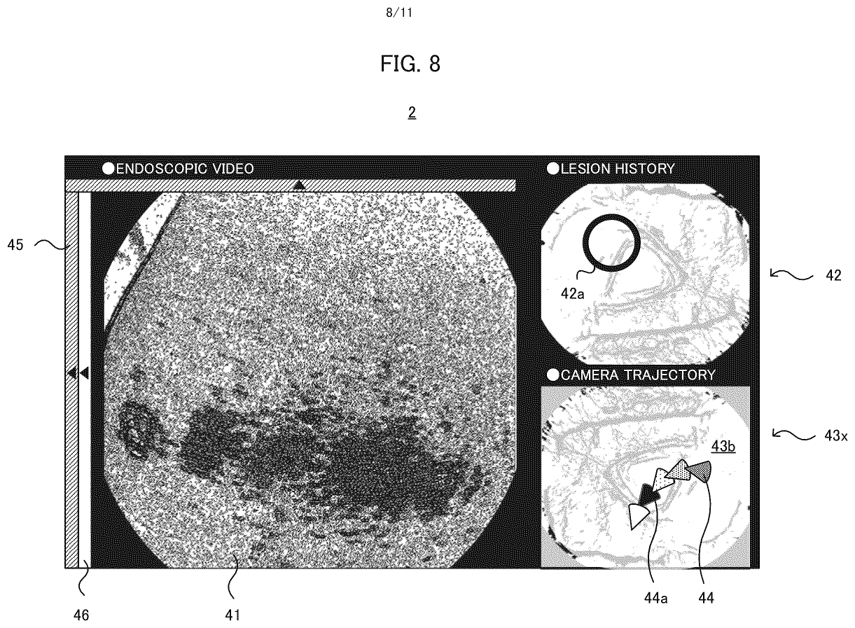

FIG. 8 shows another display example by the display device 2. In the example of FIG. 6, the camera trajectory is displayed on the intestinal tract model 43a. In contrast, FIG. 8 shows an example in which the camera trajectory is displayed on the endoscopic image. Specifically, in the camera trajectory 43x of FIG. 8, the camera marks 44 indicating the orientation and position of the endoscope camera at predetermined times are superimposed on the endoscopic image 43b. Here, as the endoscopic image 43b, an endoscopic image in a previous ideally photographing direction, e.g., an endoscopic image captured while the endoscope camera is directed in the intestinal tract direction is used. Also, in the camera trajectory 43x of FIG. 8, in addition to the camera trajectory, the ideal position of the camera is indicated by the camera mark 44a of a black cone. In this case, as the endoscopic image 43b shown in FIG. 8, an endoscopic image captured by the camera in the state of the camera mark 44a can be used. In the example of FIG. 8, since the trajectory of the endoscope camera is displayed on the actual endoscopic image, the doctor can easily perceive the ideal position of the endoscope camera.

[Display Processing]

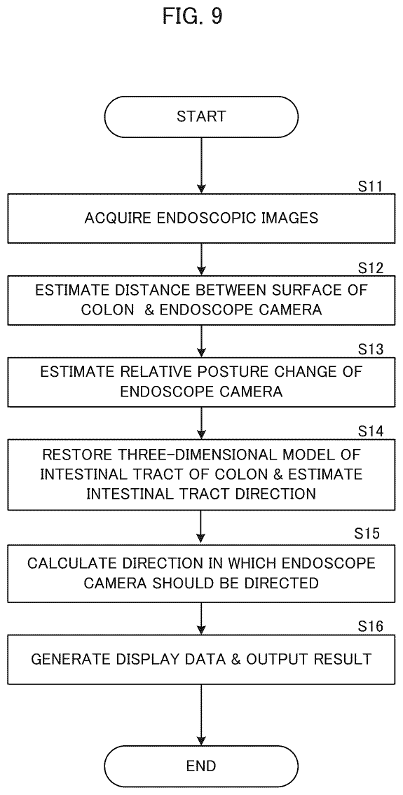

Next, display processing for performing the above-mentioned display will be described. FIG. 9 is a flowchart of processing performed by the endoscopic examination support apparatus 1. This processing is realized by the processor 11 shown in FIG. 2, which executes a pre-prepared program and operates as the elements shown in FIG. 3. This processing is performed during the endoscopic examination, i.e., during removal of the endoscope 3.

First, an endoscopic video Ic is inputted from the endoscope 3 to the interface 13. The interface 13 acquires the endoscopic images from the inputted endoscopic video Ic (step S11). Next, the depth estimation unit 21 estimates the distance between the surface of the colon and the endoscope camera from the endoscopic images using the image recognition model prepared in advance (step S12). The camera posture estimation unit 22 estimates the relative posture change of the endoscope camera from the two endoscopic images successive in time (step S13). Next, the three-dimensional restoration unit 23 performs a three-dimensional restoration process of the intestinal tract based on the distance between the surface of the colon and the endoscope camera and the relative posture change of the endoscope camera, and estimates the direction of the intestinal tract (step S14). Then, the operation direction estimation unit 24 calculates the direction in which the endoscope camera should be directed, on the basis of the relative posture change of the endoscope camera and the direction of the intestinal tract (step S15).

The display image generation unit 26 generates display data using the three-dimensional model, the relative posture change of the endoscope camera, and the direction in which the endoscope camera should be directed, and outputs the generated display data to the display device 2 (step S16). Thus, the display as shown in FIG. 5 or the like is performed. Incidentally, step S13 may be performed prior to step S12, or may be performed simultaneously with step S12.

Thus, the display image can be used to support user's decision making.

### Second Example Embodiment

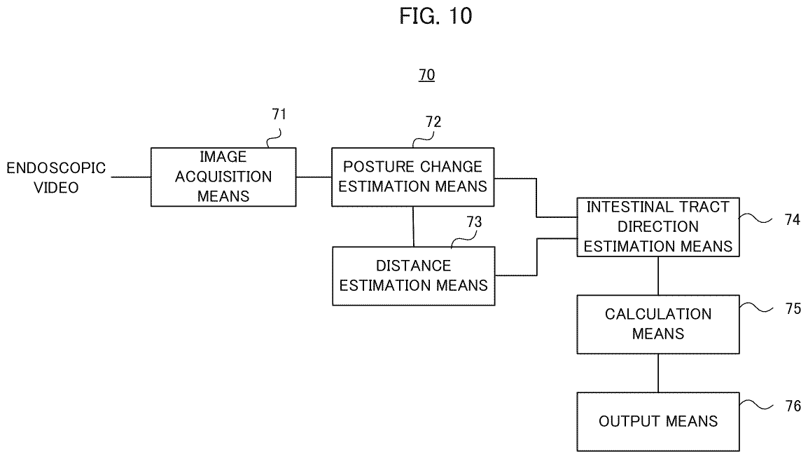

FIG. 10 is a block diagram illustrating a functional configuration of an endoscopic examination support apparatus according to a second example embodiment. The endoscopic examination support apparatus 70 includes an image acquisition means 71, a posture change estimation means 72, a distance estimation means 73, an intestinal tract direction estimation means 74, a calculation means 75, and an output means 76,

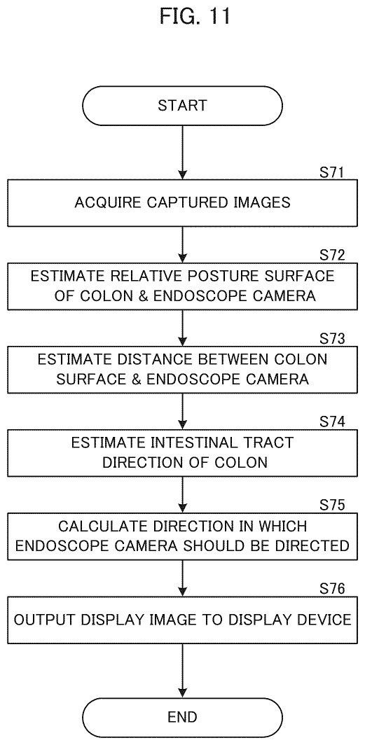

FIG. 11 is a flowchart of processing performed by the endoscopic examination support apparatus according to the second example embodiment. The image acquisition means 71 acquires captured images during removal of an endoscope (step S71). The posture change estimation means 72 estimates a relative posture change of an endoscope camera from the captured images (step S72). The distance estimation means 73 estimates a distance between a surface of colon and the endoscope camera from the captured images (step S73). The intestinal tract direction estimation means 74 estimates an intestinal tract direction of the colon based on the posture change and the distance (step S74). The calculation means 75 calculates a direction in which the endoscope camera should be directed, based on the intestinal tract direction and the relative posture of the endoscope camera (step S75). The output means 76 outputs a display image including the direction in which the endoscope camera should be directed, to a display device (step S76).

According to the endoscopic examination support apparatus 70 of the second example embodiment, during the endoscopic examination, it becomes possible to present the direction of the endoscope camera suitable for observation.

A part or all of the example embodiments described above may also be described as the following supplementary notes, but not limited thereto.

(Supplementary Note 1)

An endoscopic examination support apparatus comprising:
an image acquisition means configured to acquire captured images during removal of an endoscope;
a posture change estimation means configured to estimate a relative posture change of an endoscope camera from the captured images;
a distance estimation means configured to estimate a distance between a surface of colon and the endoscope camera from the captured images;
an intestinal tract direction estimation means configured to estimate an intestinal tract direction of the colon based on the posture change and the distance;
a calculation means configured to calculate a direction in which the endoscope camera should be directed, based on the intestinal tract direction and the relative posture of the endoscope camera; and
an output means configured to output a display image including the direction in which the endoscope camera should be directed, to a display device.

(Supplementary Note 2)

The endoscopic examination support apparatus according to Supplementary note 1, wherein the direction in which the endoscope camera should be directed is the intestinal tract direction.

(Supplementary Note 3)

The endoscopic examination support apparatus according to Supplementary note 1, wherein the posture change estimation means estimates the posture change using a machine learning model that is trained, in advance, to estimate a depth and the posture change of the endoscope camera from the endoscopic images.

(Supplementary Note 4)

The endoscopic examination support apparatus according to Supplementary note 1,
wherein the direction in which the endoscope camera should be directed includes the intestinal tract direction and a lesion direction, and
wherein the output means outputs the display image which displays the intestinal tract direction and the lesion direction in a distinguishable manner.

(Supplementary Note 5)

The endoscopic examination support apparatus according to Supplementary note 1, wherein the intestinal tract direction estimation means generates an intestinal tract model based on the posture change and the distance, and estimates the intestinal tract direction based on the intestinal tract model.

(Supplementary Note 6)

The endoscopic examination support apparatus according to Supplementary note 5, wherein the output means outputs the display image in which a trajectory of postures of the endoscope camera is superimposed and displayed on the intestinal tract model.

(Supplementary Note 7)

The endoscopic examination support apparatus according to Supplementary note 6, wherein the output means outputs the display image in which the intestinal tract model is viewed in a direction in which overlap of the postures of the endoscope camera in the trajectory is small.

(Supplementary Note 8)

The endoscopic examination support apparatus according to Supplementary note 1, wherein the output means outputs the display image in which a trajectory of postures of the endoscope camera and the direction in which the endoscope camera should be directed are superimposed and displayed on the captured image.

(Supplementary Note 9)

An endoscopic examination support method comprising:
acquiring captured images during removal of an endoscope;
estimating a relative posture change of an endoscope camera from the captured images;
estimating a distance between a surface of colon and the endoscope camera from the captured images;
estimating an intestinal tract direction of the colon based on the posture change and the distance;
calculating a direction in which the endoscope camera should be directed, based on the intestinal tract direction and the relative posture of the endoscope camera; and
outputting a display image including the direction in which the endoscope camera should be directed, to a display device.

(Supplementary Note 10)

A recording medium recording a program, the program causing a computer to execute processing of:
acquiring captured images during removal of an endoscope;
estimating a relative posture change of an endoscope camera from the captured images;
estimating a distance between a surface of colon and the endoscope camera from the captured images;
estimating an intestinal tract direction of the colon based on the posture change and the distance;
calculating a direction in which the endoscope camera should be directed, based on the intestinal tract direction and the relative posture of the endoscope camera; and
outputting a display image including the direction in which the endoscope camera should be directed, to a display device.

This application is based upon and claims the benefit of priority from the international application PCT/JP2022/029450 filed Aug. 1, 2022, the disclosure of which is hereby incorporated by reference in its entirety.

While the present disclosure has been described with reference to the example embodiments and examples, the present disclosure is not limited to the above example embodiments and examples. Various changes which can be understood by those skilled in the art within the scope of the present disclosure can be made in the configuration and details of the present disclosure.

### DESCRIPTION OF SYMBOLS

1 Endoscopic examination support apparatus
2 Display device
3 Endoscope
11 Processor
12 Memory
13 Interface
21 Depth estimation unit
22 Camera posture estimation unit
23 three-dimensional restoration unit
24 Operation direction estimation unit
25 Lesion detection unit
26 Display image generation unit
100 Endoscopic examination system

1. An endoscopic examination support apparatus comprising:
a memory configured to store instructions; and
one or more processors configured to execute the instructions to:
acquire captured images during removal of an endoscope;
estimate a relative posture change of an endoscope camera from the captured images;
estimate an intestinal tract direction of a colon based on the posture change and a distance between a surface of colon and the endoscope camera using the captured images;
calculate a direction in which the endoscope camera should be directed, based on the intestinal tract direction and the relative posture of the endoscope camera; and
output, to a display device, a display image including an indicator presenting the direction in which the endoscope camera should be directed, wherein the indicator is displayed at least one of the upper, lower, left and right ends of the captured images.

2. The endoscopic examination support apparatus according to claim 1, wherein the one or more processors are further configured to execute the instructions to:
estimate the posture change using a machine learning model that is trained, in advance, to estimate a depth and the posture change of the endoscope camera from the captured images.

3. The endoscopic examination support apparatus according to claim 1,
wherein the direction in which the endoscope camera should be directed is the intestinal tract direction of the colon.

4. The endoscopic examination support apparatus according to claim 1, wherein the one or more processors are further configured to execute the instructions to:
detect a lesion candidate from the captured images.

5. The endoscopic examination support apparatus according to claim 4, wherein the one or more processors are further configured to execute the instructions to:
wherein the direction in which the endoscope camera should be directed is the direction of the lesion.

6. The endoscopic examination support apparatus according to claim 5,
wherein the one or more processors are further configured to execute the instructions to:
output the display image which displays the intestinal tract direction and the lesion direction in a distinguishable manner.

7. An endoscopic examination support method, executed by a computer, comprising:
acquiring captured images during removal of an endoscope;
estimating a relative posture change of an endoscope camera from the captured images;
estimating an intestinal tract direction of a colon based on the posture change and a distance between a surface of colon and the endoscope camera using the captured images;
calculating a direction in which the endoscope camera should be directed, based on the intestinal tract direction and the relative posture of the endoscope camera; and
outputting a display image including an indicator presenting the direction in which the endoscope camera should be directed, wherein the indicator is displayed at least one of the upper, lower, left and right ends of the captured images, to a display device.

8. A recording medium that records a program for supporting an endoscopic examination, for causing a computer to execute:
acquiring captured images during removal of an endoscope;
estimating a relative posture change of an endoscope camera from the captured images;
estimating an intestinal tract direction of a colon based on the posture change and a distance between a surface of colon and the endoscope camera using the captured images;
calculating a direction in which the endoscope camera should be directed, based on the intestinal tract direction and the relative posture of the endoscope camera; and
outputting a display image including an indicator presenting the direction in which the endoscope camera should be directed, wherein the indicator is displayed at least one of the upper, lower, left and right ends of the captured images, to a display device.
